(19) United States
(12) Patent Application Publication
(10) Pub. No.: US 2004/0076664 A1
(43) Pub. Date: Apr. 22, 2004

(54) Chewable, soft gel, high potency vitamins and method of manufacture

(76) Inventor: Michael P. Bonura, Valley Village, CA (US)

Correspondence Address: Sanford Astor, Suite 200, 10507 West Pico Blvd., Los Angeles, CA, 90064, US

(21) Appl. No.: 10/277,771

(22) Filed: Oct. 22, 2002

(51) Int. Cl. A61K 9/48; A61K 9/20

(52) U.S. Cl. 424/465; 264/109

(57) ABSTRACT

Chewable, soft gel multi-vitamins and a method for their manufacture, particularly children's vitamins, by a low velocity, low temperature process in which pharmaceutical grade raw materials are utilized in a way that their potency is maintained into the finished product.

### BACKGROUND OF THE INVENTION

Vitamins are a multi-billion dollar business. Children's vitamins form a significant part of the vitamin market. Making a vitamin which children will take has been a problem faced by the industry. That is why there are vitamins such as Flintstones, Bugs Bunny, Scooby Doo, Pokemon, Sesame Street, one A Day, Popeye, Zippy Zoo, all of which are essentially the same vitamin mixture with different licensing agreements based on the characters into which the vitamins are shaped. These standard vitamins use low grade materials in a gelatin based chalky powder compressed by a high velocity molding/tablet machine with artificial colors. A heat process allows the manufacturers to mass produce huge numbers of these low grade vitamins. The heat process now used in these high velocity machines by these manufacturers depletes up to about 85% of the nutritional value of the raw materials.

### SUMMARY OF THE INVENTION

Applicant's invention comprises manufacturing vitamins, particularly children's vitamins, by a low velocity, low temperature process in which pharmaceutical grade raw materials are utilized in a way that their potency is maintained into the finished product. The multi-vitamin end product of this invention is a chewable, soft gel, high potency multi-vitamin comprising a plurality of vitamins, minerals and/or anti-oxidants, in which a “serving” of one or more of the vitamins provides 100% of the RDA (Recommended Daily Allowance. In addition, the vitamin has the soft and chewy consistency of common jelly beans and is pleasant to take, being particularly usable as a vitamin for children. The recommended “serving” usually consists of several chewable, soft gels, the vitamin ingredients all being pharmaceutical grade, microcrystalline encapsulated powder, in a lecithin base, with a natural sweetener, together with other trace elements.

The exact formula for each soft gel may vary depending upon the exact nutritional blend desired but the method of manufacture is applicable to any desired formula, since the method of manufacture maintains the full potency of the raw materials used.

### OBJECTS OF THE INVENTION

Accordingly, several objects and advantages of the invention are as follows:

It is an object of the present invention to provide a pharmaceutical grade chewable, soft gel, high potency vitamin which is appealing to take, particularly for children.

Another object of the invention is to provide a method for the manufacture of chewable, soft gel, high potency vitamins, in large quantities, while maintaining the full potency of the raw materials used.

These, as well as other objects of the invention, will become obvious from the following description in which:

### DETAILED DESCRIPTION OF THE INVENTION

The vitamins: The chewable, soft gel multi-vitamins produced by this invention comprise a plurality of active ingredients selected from vitamins, minerals and anti-oxidants, having 100% of RDA per serving, each serving consisting of one or more chewable, soft gels. Although the preferred shape of the soft gels is that of a jelly bean, any other shape desired may also be made. All ingredients are pharmaceutical grade, in a microcrystalline, encapsulated powder, in a lecithin base, with a natural sweetener, together with trace amounts of other ingredients which can be one or more of beeswax, carnauba wax, protein glaze, dextrose, fruit extracts, cellulose, masking agent, and color. The natural sweetener can be sweeteners such as kiwi extract, pure vanilla extract and/or xylitol.

The method of manufacture:

1) The manufacturing process begins by mixing ingredients in a stainless steel mixing container to form the centers of the soft gels. The ingredients comprise lecithin in a granular form, one or more of beeswax, dextrose, carnauba wax, corn syrup, corn starch, pure vanilla extract, a natural sweetener of Kiwi pure fruit extract and/or xylitol and the active ingredients selected from vitamins, minerals and anti-oxidants. About one-fourth of the total active ingredients, selected from vitamins, minerals and anti-oxidants, are added in this first step. The stainless steel mixing container has slow turning mixing blades which blend this mixture at a very low velocity, at a low temperature, not to exceed 80 degrees F, for about 18-24 hours, until the mixture becomes a soft, chewy thick slurry.

2) The slurry is put into molding trays, each mold unit being any desired shape (such as the shape of a jelly bean). The molding trays are put into an air pressure, drying, clean room for about 24-36 hours, with a constant air pressure, under about 80 degrees F, where the soft gel centers harden to a chewable, soft gel consistency.

3) The soft gel centers are taken out of the molding trays and are put into low rpm turning drums. The soft gels are first sprayed with a light mist of natural spring water and naturals sweeteners so that they become tacky on the outside surface only. About another one-fourth of the active ingredients, selected from vitamins, minerals and anti-oxidants, are added into the turning drums, which stick to the tacky outer surface of the soft gels.

4) Process 3 is repeated two more times to add a total of three layers of the active ingredients. Each time a mist of natural spring water is added to give the active ingredients something to stick to, and to keep the consistency layered. The third time a trace amount of coloring agent is added and all the desired active ingredients are now in each soft gel.

5) All of the soft gels are put back into molding trays and put into the air pressure, drying room for 18-24 hours, to allow the soft gels to dry.

6) The soft gels are now put into a panning room into small rotating drums, at about less than 80 degrees F, with a natural protein glaze, carnauba wax, natural sweeteners and natural colors to create the coating or shell and polish the soft gels to their final stage, where they have a shiny finish. A serving of one or more soft gels now contain 100% of the RDA in every category of active ingredient desired, in a non-gelatin, chewable, soft gel, multi-vitamin.

A specific preferred process is set forth below:

1) In a 2040 pound stainless steel mixing container are placed 2000 pounds of ingredients made up of, by weight, 34-38% lecithin, in a granular form, to which is added 13-15% beeswax, 8-10% carnauba wax, 3-5% dextrose and 20-25% active ingredients, consisting of vitamins, minerals and antioxidant powders. About one-fourth of the total of active ingredients of the finished product are added in this first step. Also added are 2-3% corn syrup, 1-2% corn starch, 1-2% citric acid and trace amounts of pure vanilla extract, Kiwi pure fruit extract and Xylitol. This mixture is put into an oven at less than 80 degrees F, preferably 77 degrees F., and blended at very low velocity until the mixture reaches a soft, chewy consistency. This will take from 18 to 30 hours. This step creates the soft gel “centers”.

2) The mixture is then put into molding trays, each tray having a plurality of molds the shape of a jelly bean, each mold able to hold about 2.2 grams of ingredients. In this step, about 1.28 grams of mixture are placed in each jelly bean shaped mold.

3) The molding trays are now put into air pressure drying rooms, where the trays remain for 34-48 hours at a constant air pressure which is low in temperature (below 80 degrees F), preferably 77 degrees F. The drying rooms are clean rooms, sealed to outside conditions. This drying process allows the soft gel “centers” to reach a solid workable state, however they remain having a chewy soft gel consistency.

4) The molding trays are then brought into a panning room where at a low velocity and low temperature (below 80 degrees F),preferably 77 degrees F, the “centers” are sprayed with a light mist of natural spring water so that they become tacky on the outside surface. Then an additional one-fourth of the active ingredient vitamin, mineral, antioxidant powders are layered onto the “centers” by turning the “centers” in low rpm turning drums where the active ingredients stick to the tacky surface of the “centers”. This process is then repeated, two more times, creating layers, until the total desired amount of vitamin, mineral, antioxidant mixture is layered onto the “centers”, creating the soft gels.

5) The soft gels are put back into the molding trays and the trays are put back into the air drying room for 10-24 hours to insure that the vitamin, mineral, antioxidant powders and water mist have a chance to dry.

6) The molding trays are then brought back into the panning room where the soft gels are put back into the low velocity turning drums to add the coating or shell comprising 70% by weight of a protein glaze, and 30% by weight a natural sweetener, such as xylitol. A serving of the soft gels now contain 100% of the RDA in every category of vitamins, minerals and antioxidants desired. This may be one or more soft gels depending upon what the consumer wants to take.

Any combination of known vitamins, minerals and antioxidants may be used, depending upon the desire of the manufacturer. A preferred composition of vitamins, minerals and antioxidants, applicable to children, is shown below in Table 1.

TABLE 1

| Ingredient | Amount per soft gel | |
|---|---|---|
| Vitamin A (as Palmitate) | 5,000 | IU |
| Vitamin E (Natural d-alpha tocopherol) | 50 | IU |
| Vitamin C (Calcium Ascorbate) | 100 | mg |
| Citrus Bioflavonoid Complex | 50 | mg |
| Zinc (as Monomethionat) | 12 | mg |
| Selenium (as Sodium Selenate) | 30 | mcg |
| Vitamin B-1 ( Thiamin) | 5 | mg |
| Vitamin B-2 (Roboflavin) | 5 | mg |
| Vitamin B-3 (Niacin) | 5 | mg |
| Niacinamide | 5 | mg |
| Vitamin B-5 (Calcium Paniothenate) | 5 | mg |
| Vitamin B-6 (Pyridoxine HCL) | 5 | mg |
| Vitamin B-12 (Methylcobalamin) | 50 | mcg |
| Folic Acid | 200 | mcg |
| Biotin | 100 | mcg |
| Vitamin D (as vitamin D-3) | 200 | IU |
| Vitamin K (as vitamin K-1) | 25 | mcg |
| Calcium (as Calcium Citrate) | 400 | mg |
| Magnesium (as Magnesium Oxide) | 200 | mg |
| Potassium (as Potassium Chloride) | 99 | mg |
| Manganese (as Manganese Gluconate) | 2 | mg |
| Iron (as Ferrous Gluconate) | .8 | mg |
| Chromium (as GTF) | 100 | mcg |
| Copper (as Copper Gluconate) | 1 | mg |
| Iodine (Potassium Iodide) | 125 | mcg |
| Molybdenum (as Sodium Molybdate) | 50 | mcg |
| Choline | 250 | mg |
| Inositol | 250 | mg |
| Fiber (Chicory) | 50 | mg |

The resulting vitamin, mineral, antioxidant product contains 100% nutrition for children in a non-gelatin base using natural sugars, that taste good and are appealing for children to take.

Any combination of vitamins, minerals or anti-oxidants alone or in any combinations may be prepared depending upon the purpose for which the soft gels are to be used. The resulting soft gels are soft and chewy, very similar to a jelly bean.

Having thus described the invention, I claim:

1: A method for manufacturing a chewable, soft gel multi-vitamin comprising:
a) mixing in a container having slow moving mixing blades, a combination of lecithin, one or more active ingredients selected from vitamins, minerals and anti-oxidants, beeswax, carnauba wax, dextrose, corn syrup, corn starch, citric acid and one or more trace elements selected from vanilla extract, kiwi extract and xylitol, mixing the selected ingredients at a temperature, not to exceed 80 degrees F, for about 18-24 hours;
b) putting the mixed ingredients into molding trays having individual mold units, placing the molding trays in an air pressure drying room for about 24-36 hours, below about 80 degrees F where the mixture hardens to a chewy, soft gel;
c) putting the soft gels into a slow turning drum, spraying the soft gels one or more times with a mist of water, adding additional amounts of active ingredients selected from vitamins, minerals, and antioxidants, which stick to the surface of the soft gels;
d) putting the soft gels back into molding trays, placing the molding trays in an air pressure drying room for about 18 to 24 hours, to allow the soft gels to dry;
e) putting the soft gels into rotating drums, below about 80 degrees F, and adding a protein glaze and natural sweetener to create the outer shell of the soft gels.

2: The method of claim 1 in which a combination of vitamins, minerals and anti-oxidants are utilized.

3: The method of claim 1 in which beeswax, dextrose, glycerin, vanilla extract, kiwi extract and xylitol are all utilized.

4: The method of claim 1 in which a trace amount of color is added to step c.

5: The method of claim 1 in which the lecithin, vitamins minerals and anti-oxidants are all in a powdered form.

6: The method of claim 1 in which the soft gels are in the shape of jelly beans.

7: The method of claim 1 in which the ingredients in step a are mixed at 77 degrees F.

8: The method of claim 1 in which drying step b takes place at 77 degrees F.

9: The method of claim 1 in which the water spray is a natural spring water.

10: A chewable, soft gel, multi-vitamin comprising active ingredients selected from vitamins, minerals and antioxidants, in a lecithin base, containing one or more of beeswax, carnauba wax, dextrose, corn syrup, corn starch, citric acid and trace amounts of one or more of vanilla extract, kiwi pure fruit extract and xylitol.

11: The soft gel of claim 10 comprising vitamins, minerals and antioxidants.

12: The soft of claim 10 comprising all of beeswax, carnauba wax, dextrose, corn syrup, corn starch, citric acid and trace amounts of one or more of vanilla extract, kiwi pure fruit extract and xylitol.

13: The soft gel of claim 10 further comprising a shell of protein glaze and natural sweetener.

14: The soft gel of claim 13 in which the sweetener is selected from vanilla extract, kiwi pure fruit extract and xylitol.

15: The soft gel of claim 13 further comprising a coloring agent.

16: The soft gel of claim 10 in which the active ingredients comprise:

| | | |
|---|---|---|
| Vitamin A (as Palmitate) | 5,000 | IU |
| Vitamin E (Natural d-alpha tocopherol) | 50 | IU |
| Vitamin C (Calcium Ascorbate) | 100 | mg |
| Citrus Bioflavonoid Complex | 50 | mg |
| Zinc (as Monomethionat) | 12 | mg |
| Selenium (as Sodium Selenate) | 30 | mcg |
| Vitamin B-1 ( Thiamin) | 5 | mg |
| Vitamin B-2 (Roboflavin) | 5 | mg |
| Vitamin B-3 (Niacin) | 5 | mg |
| Niacinamide | 5 | mg |
| Vitamin B-5 (Calcium Paniothenate) | 5 | mg |
| Vitamin B-6 (Pyridoxine HCL) | 5 | mg |
| Vitamin B-12 (Methylcobalamin) | 50 | mcg |
| Folic Acid | 200 | mcg |
| Biotin | 100 | mcg |
| Vitamin D (as vitamin D-3) | 200 | IU |
| Vitamin K (as vitamin K-1) | 25 | mcg |
| Calcium (as Calcium Citrate) | 400 | mg |
| Magnesium (as Magnesium Oxide) | 200 | mg |
| Potassium (as Potassium Chloride) | 99 | mg |
| Manganese ( as Manganese Gluconate) | 2 | mg |
| Iron (as Ferrous Gluconate) | .8 | mg |
| Chromium (as GTF) | 100 | mcg |
| Copper (as Copper Gluconate) | 1 | mg |
| Iodine (Potassium Iodide) | 125 | mcg |
| Molybdenum (as Sodium Molybdate) | 50 | mcg |
| Choline | 250 | mg |
| Inositol | 250 | mg |
| Fiber (Chicory) | 50 | mg |
